(19) United States
(12) Patent Application Publication
(10) Pub. No.: US 2017/0086869 A1
(43) Pub. Date: Mar. 30, 2017

(54) FLEXIBLE ARTICULATED DEVICE

(71) Applicant: LARYNGOPORT LTD., Jerusalem (IL)

(72) Inventor: Revital ROZENFELD, Nes Ziyona (IL)

(21) Appl. No.: 15/310,772

(22) PCT Filed: May 11, 2015
(86) PCT No.: PCT/IL2015/050491

§ 371 Date: Nov. 13, 2016

(30) Foreign Application Priority Data
May 13, 2014 (IL) 232605

(51) Int. Cl. A61B 17/29; A61B 34/00

(52) U.S. Cl. CPC A61B 17/29; A61B 34/71; A61B 2017/2929; A61B 2017/00327

(57) ABSTRACT

The present innovation pertains to a device and method for enabling movement of a flexible medical device.

### FIELD OF THE INVENTION

The present invention relates methods and devices for enabling movement of a working end of a laryngeal microsurgical device.

### BACKGROUND OF THE INVENTION

In laryngoscopy surgical procedures, an elongate shaft of a surgical device is inserted through the mouth to position a distal end of the shaft at a surgical site. These kind of procedures are usually performed with rigid shafts that are less suitable to accommodate the tortuous pathway of the body lumen. These tools can be used to engage and/or treat tissue in a number of ways to achieve a therapeutic effect.

Current laryngoscopy devices do not utilize articulating effectors to provide the user with more control over the orientation of the working end of the instrument. Integration of the controls for articulating, as well as actuating, tend be complicated by the size constraints of the relatively small pathway through which they have to be inserted. Generally, the control motions are all transferred through the shaft as longitudinal translations, which sometimes can interfere with the control of the shaft.

A number of articulating medical instruments and methods are known in the art. For example: U.S. Pat. No. 8,308,738 describes methods and devices provided for controlling movement of a working end of a surgical device, and in particular for performing various surgical procedures using an instrument having an end effector that can be articulated relative to an elongate shaft of the device. In certain embodiments, the end effector can also optionally rotate relative to the elongate shaft of the device, and/or the shaft can rotate relative to a handle of the device. While these instruments are rigid and relatively easy to control, complications arise with current devices purported to be able to be used for multiple actions. Accordingly, there remains a need for improved methods and devices for controlling movement of a working end of a laryngoscopy surgical device.

### SUMMARY OF PREFFERED EMBODIMENTS

It is an object of the present invention to provide a surgical device comprising a three-bar-linkage retraction mechanism, said mechanism comprises a distal element 160, link 150 and a flexible elongated (endless) shaft 110 provided proximally to the three link bar, wherein the link is interconnected to the shaft, by mean of a rigid clevis 140, which is configured to enable simultaneously three degrees of freedom namely rotation, actuation and articulation.

It is a further object of the present invention to provide the aforementioned device wherein the rigid clevis is provided with a guide 172 configured for guiding the actuator wire 180. Furthermore, the guide 172 is configured by having at least a section of a concaved-parabolic nozzle-like shape, configured for guidance of the wire and fixture at the nozzles throat 175.1.

It is another object of the present invention to provide the aforementioned device wherein the rigid clevis is provided with a guide 172 configured for separating the actuator wire 180 from link 150.

It is a further object of the present invention to provide the aforementioned device wherein the guide is configured for guiding and separating the actuator wire 180.

It is another object of the present invention to provide the aforementioned device wherein the rigid clevis is coupled to the distal end of an elongate flexible shaft and to the proximal end of the end effector. The device also comprises an actuation wire extending along the elongate shaft, configured to be linearly reciprocated in parallel with the main longitudinal axis of the shaft. It is also configured to translate, along a longitudinal axis of the elongate shaft, actuation of the effector.

It is an object of the present invention to provide the aforementioned device wherein at least a portion of the clevis is characterized by a concaved-parabolic nozzle like shape. Furthermore, at least a portion of said guide is characterized by a concaved-parabolic nozzle-like shape.

It is a further object of the present invention to provide the aforementioned device wherein at least one distal portion of the shaft comprises a coiled rotation-enabling shaft. This coiled shaft is accommodated within the main flexible shaft and a distal part of it is accommodated within the clevis. The coiled shaft id also connected with a link 150.

It is another object of the present invention to provide the aforementioned device wherein an actuator wire is accommodated within the coil system.

is a further object of the present invention to provide the aforementioned device wherein is configured with an n-bar linkage, n is integer equal or greater than 3. The device can include parts that are flexible, rigid and any combination thereof.

It is an object of the present invention to provide the aforementioned device wherein the device comprises a mechanism adapted to control at least three degrees of freedom, selected from a group consisting of activation of the element, articulation of distal end of the element, rotation of the distal end of the element, and any combination thereof.

It is another object of the present invention to provide the aforementioned device wherein the mechanism is enabled by the clevis.

It is an object of the present invention to disclose the aforementioned method for enabling movement in a flexible shaft, wherein the method comprises
a. obtaining a surgical device comprising a three-bar-linkage retraction mechanism, that comprises a distal element 160, a link 150 and a flexible elongated (endless) shaft 110 provided proximally to the three link bar, wherein the link is interconnected to the shaft, by mean of rigid clevis 140, thereby enabling simultaneously three degrees of freedom namely rotation, actuation and articulation and
b. operating said device

It is an object of the present invention to disclose the aforementioned method wherein said rigid clevis is provided with a guide 172 configured for guiding said actuator wire 180. The guide 172 is configured to have, at least a section, of a shape characterized by a concaved-parabolic nozzle-like form. This shape is configured for guidance of the wire and fixture at the nozzles throat.

It is another object of the present invention to disclose the aforementioned method wherein the rigid clevis is provided with a guide 172 configured for separating the actuator wire 180 from link 150. The guide is also configured for guiding and separating the actuator wire 180.

It is a further object of the present invention to disclose the aforementioned method. wherein rigid clevis is coupled to the distal end of the elongate flexible shaft and to the proximal end of the end effector.

It is another object of the present invention to disclose the aforementioned method wherein the device further comprises an actuation wire extending along the elongate shaft, configured to be linearly reciprocated in parallel with the main longitudinal axis of the shaft. Furthermore, the actuation wire extends through the elongate shaft, and it is configured to translate, along a longitudinal axis of the elongate shaft, actuation of the effector.

It is an object of the present invention to disclose the aforementioned method wherein at least a portion of the clevis is characterized by a concaved-parabolic nozzle-like shape.

It is another object of the present invention to disclose the aforementioned method wherein at least a portion of said guide is characterized b a concaved-parabolic nozzle-like shape.

It is a further object of the present invention to disclose the aforementioned method wherein at least one distal portion of the shaft comprises a coiled rotation-enabling shaft, and the flexible coiled rotation-enabling shaft is accommodated within the main flexible shaft.

It is an object of the present invention to disclose the aforementioned method wherein a distal part of said flexible coded rotation-enabling shaft is accommodated within the clevis.

It is another object of the present invention to disclose the aforementioned method wherein the flexible coiled rotation-enabling shaft is connected with a link 150.

It is a further object of the present invention to disclose the aforementioned method wherein an actuator wire is accommodated within the coil system.

It is an object of the present invention to disclose the aforementioned method wherein the device is configured with an n-bar linkage, 11 is integer equal or greater than 3.

It is another object of the present invention to disclose the aforementioned method wherein parts of the device are flexible, rigid and any combination thereof.

It is an object of the present invention to disclose the aforementioned method of operating a flexible laryngoscopy device, method comprises steps of
a. inserting the distal end of said laryngoscopy device into the mouth of the patient wherein said steps further comprise
b. articulating distal end of the device by means of retracting said coiled shaft of device;
c. rotating distal end of the device by means of rotating said coiled shaft;
d. activating end effector by means of said actuation wire.

### BRIEF DESCRIPTION OF THE DRAWINGS

The invention is now described with reference to the drawings, wherein

FIGS. 1a-b illustrates views (not to scale) the construction of the distal end of a laryngoscopy device according to an embodiment of the invention;

FIG. 2 is an exploded view (not to scale) of the distal end of a laryngoscopy device according to an embodiment of the invention;

FIG. 3 illustrates the clevis (not to scale) and the guide according to an embodiment of the invention.

FIG. 4 illustrates the difference in configuration between a three-bar linkage and a five-bar linkage.

FIG. 5 illustrates the method of operating the device.

### DETAILED DESCRIPTION OF THE PREFFERED EMBODIMENTS

Certain exemplary embodiments will now be described to provide an overall understanding of the principles of the structure, function, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The instrument is configured for containing or carrying medical tools and/or surgical objects at the distal segment. Optionally, medical tools and/or surgical objects (hereinafter, referred to collectively as “medical tools”) are contained within the distal segment. Alternatively or additionally, one or more medical tools are attached to an end of the distal segment. Alternatively or additionally, the medical tool(s) can be inserted into the distal portion during maneuvering thereof and deployed when needed. Alternatively or additionally, a conduit or the like is inserted through the proximal and distal segments for injection of medication or collecting of tissue samples.

The present invention provides methods and devices for enhancing control on movement of a working end of a surgical device, and in particular for performing various surgical procedures using an instrument having an end effector that can be articulated relative to a flexible elongate shaft of the device.

The present invention provides a flexible shaft with a novel configuration. The novel device described herein has a flexible configuration shaft enabling three degrees of freedom to be achieved for rotation, actuation and articulation independent of each other.

In certain embodiments, the end effector can rotate relative to the elongate shaft of the device, and/or the shaft can rotate relative to a handle of the device. Articulation and rotation of the end effector will allow the end effector to be positioned at various locations during a surgical procedure, thereby providing the user with precise control over the end effector. Furthermore, in the present invention, the novel clevis provides further control on the flexible shaft, while actuating, rotating and articulating, in an independent way. A person skilled in the art will appreciate that the present invention has application in laryngoscopy procedures.

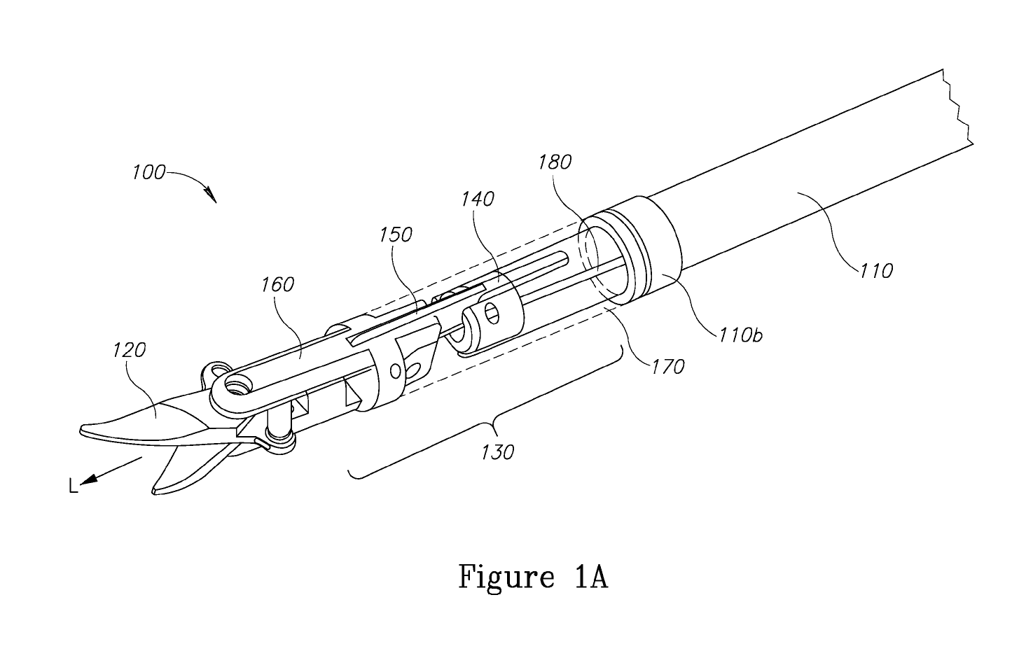

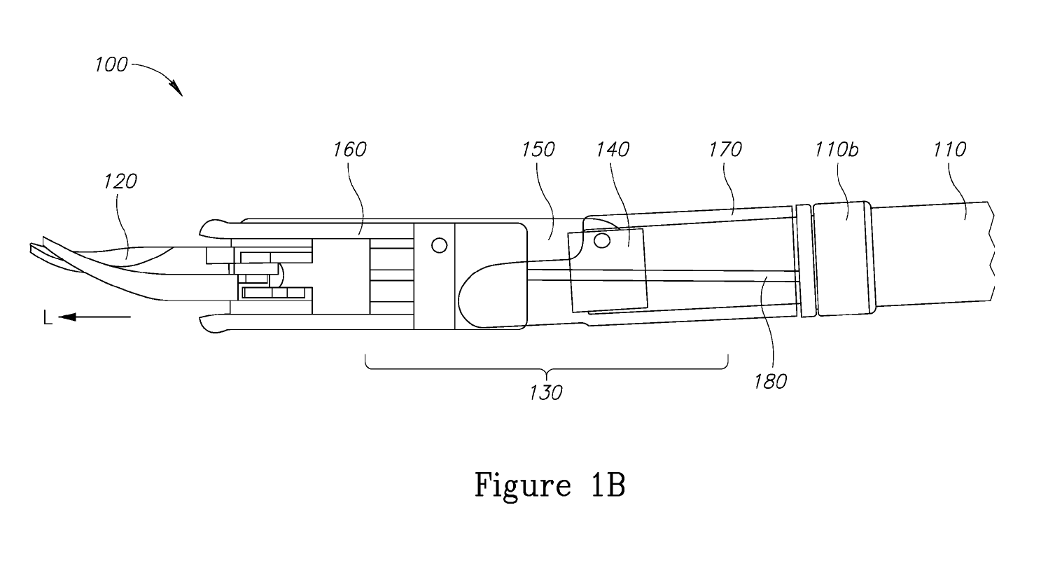

FIGS. 1a-b illustrates one explanatory embodiment of an insertion portion 100 of an either manually and/or mechanically articulating flexible device. The insertion portion 100 is preferably configured to be inserted into a patient's body, and it has rigid and flexible portions. As shown, the insertion portion 100 generally comprises a hollow flexible elongate shaft 110 coupled to a distal end 110b thereof by a three-bar linkage/clevis part 130, ending with a working end or end effector 120. While the end effector 120 can have various configurations, in the illustrated embodiment the end effector 120 is in the form of scissors having opposed jaws that are pivotally coupled to one another. The three-bar linkage/clevis 130 allows the end effector 120 to be oriented at an angle relative to a longitudinal axis L of the elongate shaft 110. The device is also configured to allow the end effector 120 to rotate relative to and about the longitudinal axis L of the elongate shaft 110. An actuation wire 130 goes through the hollowed coiled shaft, via the clevis, to the end effector.

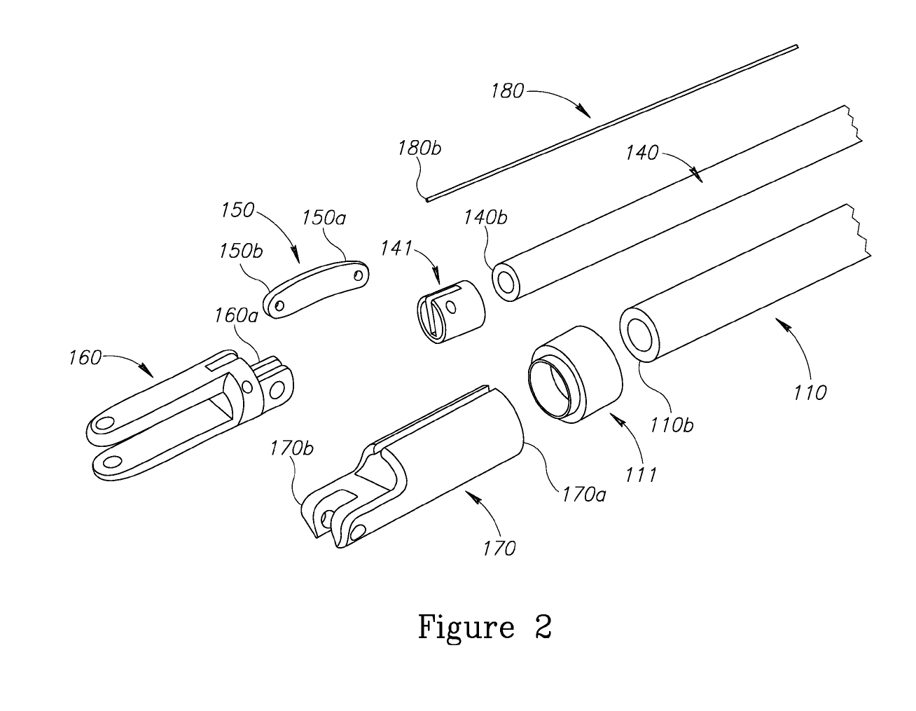

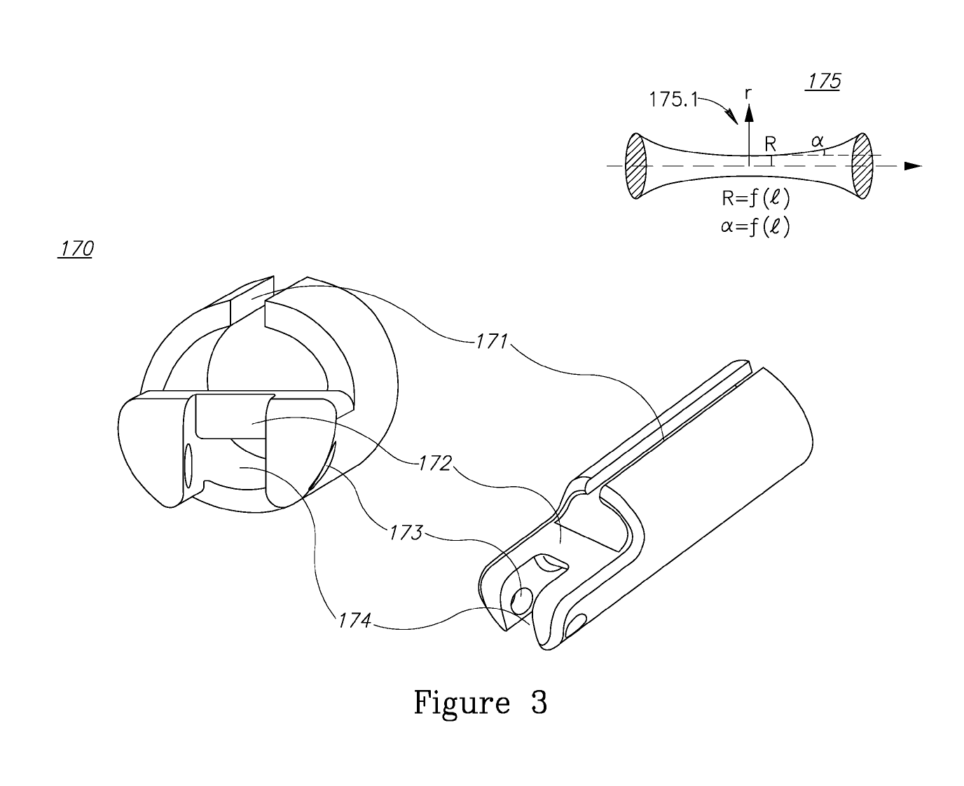

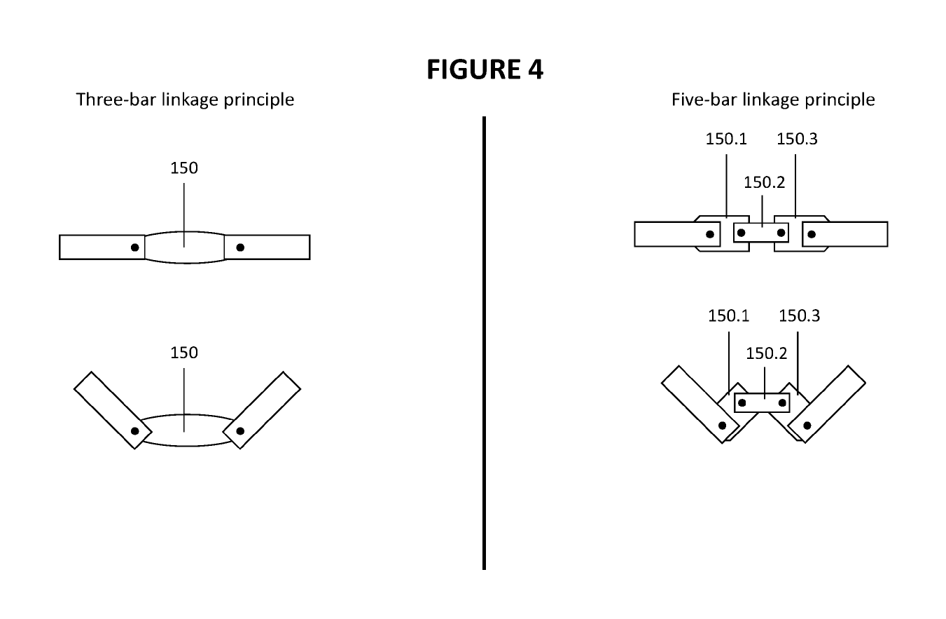

FIG. 2 illustrates one detailed explanatory embodiment. The three-bar linkage is composed by 4 distinct parts pivotally linked to each other: the coiled shaft 140, the second link 150, the end effector base 160 and the clevis 170. Rotation of the end effector is allowed by the coiled shaft 140, which is accommodated within the elongated flexible hollowed shaft 110. The distal end of the coiled shaft 140 is pivotally coupled to the clevis 170. So when the coiled shaft rotates, also the whole end (comprising of the clevis, the three-bar linkage and the end effector) turn as well. Each link can have a variety of configurations, but in an exemplar embodiment the first link is the coiled shaft 140. The second link 150 have a generally elongate shape and the third link 160 is in the form of an elongate rod or bar. The coiled shaft 140, is coupled in its distal end 140b to an adaptor 141. Adaptor 141 is coupled by a pin to a proximal end of the second link 150a. The distal end 150b of the second link 150 is coupled by a pin to a proximal end 160a of the effector base 160. The distal end 110b of the flexible shaft 110 is coupled to a rotating adaptor 111 which is successively coupled to the proximal end 170a of the clevis 170. The distal end of the clevis 170b is coupled by a pin to the proximal end 160a of the base of the end effector 160. The actuation wire 180 is accommodated within the coiled shaft 140 into the clevis 170, being separated from the second link 150 by the guide 172 (in FIG. 3). From there, the distal end 180b of the actuation wire 180 goes into the end effector 160, in which different types of tools can be configured, as shown in FIGS. 1a-b 120. The particular location, at which the base of the end effector 160 mates to the second link 150 and the clevis 170, is pivotally mated at a location that will allow the base of the en effector 160 to articulate relative to the coil system 140. This occurs when the coil system 140 is retracted.

FIG. 3 illustrates the configuration of the clevis 170. In the upper level of the clevis there is a slit 171 in which the second link 150 can move when the coil shaft 140 is being retracted. Before the distal part of the clevis there is a guide 172 that is responsible for separating the zone where the actuation wire passes 174 and the zone where the second link 150 moves. On the distal part of the clevis there are the slots 173 for the insertion of the connecting pins between the clevis 170 and the effector 160. The guide 172 is configured having, by at least a section of a shape characterized as a concaved-parabolic nozzle-like form. Exemplary diagram 175, is an idealized version of such a nozzle-like form. It is herein acknowledged that the guide section may be configured in variations of the concaved-parabolic nozzle-like form. The nozzle-like shape is configured for guidance of the actuation wire and facilitates longitudinal motion of the wire but prevents side to side motion of same.

FIGA illustrates a possible configuration of the second link 150. Usually, the second link in a three-bar linkage is made of a single, elongated piece, which is pin connected to the other links. The novel configuration of the second link comprises the division of the second link 150 into three distinct parts 150.1, 150.2, 150.3, connected to each other by pins. This configuration enables a higher articulation angle of the end effector when needed.

As indicated above, the embodiment disclosed herein for enabling movement of a working end of a surgical device is used in a variety of laryngoscopy surgical procedures. In one exemplary laryngoscopy procedure, a surgical device comprising three-bar-linkage retraction mechanism, in which said mechanism comprises a distal element 160, a link 150 and a flexible elongated shaft 110 provided proximally to the three link bar, wherein the link is interconnected to the shaft, by mean of a rigid clevis 170. Further comprising the device a flexible coiled rotation-enabling shaft 140 that is accommodated within the main flexible shaft 110 and part of its distal end is accommodated within the clevis 170. An actuation wire 180 accommodated within the coiled shaft 140 extends through the clevis 170, via the guide 172, into a part of the end effector 160.

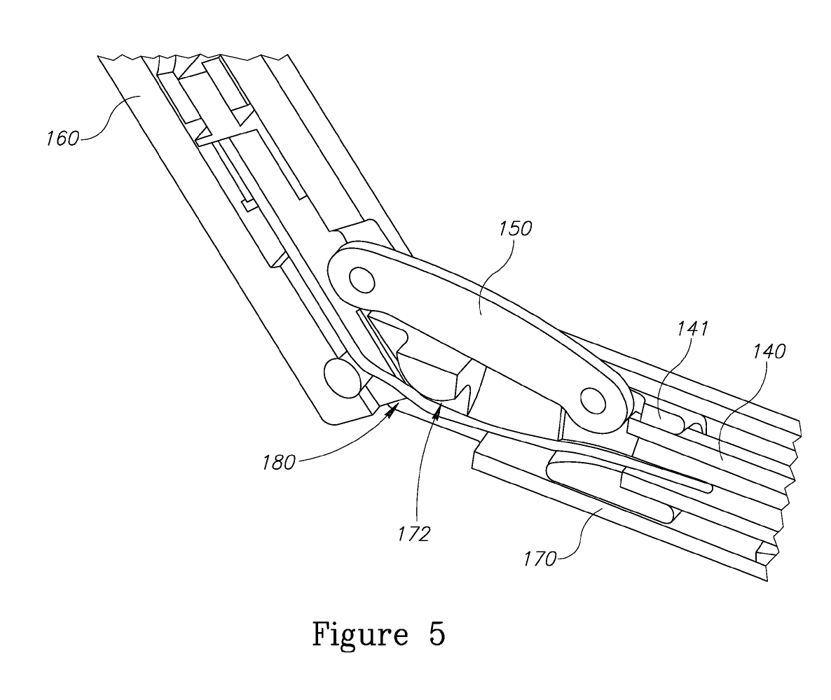

Reference is now made to FIG. 5 which is an illustration of the method of operating the device. The method comprises steps of:

Articulation
a. Retraction of coiled shaft 140, within clevis 170;
b. Retraction of adaptor 141 by coiled shaft 140;
c. Second link 150 is pulled back by adaptor 141;
d. Effector 160 is pulled up by second link 150, enabled by interconnection with clevis
   170;

Rotation
a. Rotation of coiled shaft 140, within main shaft 110 (not shown);
b. Rotation of adaptor 141 by coiled shaft 140;
c. Second link 150 is rotated by adaptor 141;
d. Effector 160 is rotated by second link 150;
e. Second link 150 rotates clevis 170, which is enabled to rotate by rotating adaptor
   111 (not shown);

Actuation
a. Retraction of actuation wire 180 activates the end effector mechanism; Three degrees of freedom
a. Actuation wire 180 is retracted within the coiled shaft 140;
b. Actuation wire 180 is guided through the clevis by guide 172;
c. Guide 172 avoids interaction between actuation wire 180 and articulation mechanism.

Retraction of the coil shaft is translated along a longitudinal axis of the elongate shaft to cause a three-bar linkage to laterally articulate the end effector in a direction perpendicular to a longitudinal axis of the elongate shaft to allow the end effector to be angularly oriented relative to the elongate shaft.

Actuation of the device is achieved by the actuation wire which, thanks to the guide in the clevis, is not affected and does not affect the rotation and articulation.

In order to achieve stabilization of the flexible shaft during all the possible actions (rotation, actuation, articulation) a rigid clevis is positioned at the distal end of the flexible shaft. The actuation wire passes through the clevis via a specific track created by the guide, separating it from the three-bar linkage, when actuation is desired. Also, for the same reason, actuation is not disturbed when articulation is in play. The clevis is connected to the main shaft by a turning adaptor which enables the rotation of the clevis when the coiled shaft is in action. Once the end effector is positioned as desired, the end effector can be used to perform a surgical procedure.

In light of the above, the advantages of the new configuration of the present invention include: usage of a flexible shaft that enables three degrees of freedom in an independent matter: rotation, actuation and articulation.

In the foregoing description, embodiments of the invention, including preferred embodiments, have been presented for the purpose of illustration and description. They are not intended to be exhaustive or to limit the invention to the precise from disclosed. Obvious modifications or variations are possible in light of the above teachings. The embodiments were chosen and described to provide the best illustration of the principals of the invention and practical application, and to enable one ordinary skill in the art to utilize the invention in various embodiments and with various modifications as are suited to the particular use contemplated. All such modifications and variations are within the scope of the invention as determined by the appended claims when interpreted in accordance with the breadth they are fairly, legal and equitably entitled.

1.-41. (canceled)

42. A surgical device comprising a three-bar-linkage retraction mechanism, said mechanism comprises a distal element, link and a flexible elongated (endless) shaft provided proximally to said three link bar, wherein said link is interconnected to said shaft, by mean of a rigid clevis, said rigid clevis configured to enable simultaneously three degrees of freedom namely rotation, actuation and articulation.

43. The device of claim 42, wherein said rigid clevis is provided with a guide configured for guiding said actuator wire.

44. The device of claim 43, wherein said guide configured by having at least a section of a concaved-parabolic nozzle-like shape.

45. The device of claim 44, wherein said nozzle-like shape is configured for guidance of the wire and fixture at the nozzles throat.

46. The device of claim 42, wherein said rigid clevis is provided with a guide configured for separating said actuator wire from link.

47. The device of claim 43 wherein said guide is configured for guiding said actuator wire.

48. The device of claim 43 wherein said guide is configured for separating said actuator wire.

49. The device of claim 42, wherein said rigid clevis is coupled to the distal end of said elongate flexible shaft and to the proximal end of said end effector.

50. The device of claim 42, further comprising said actuation wire extending along said elongate shaft, configured to be linearly reciprocated in parallel with the main longitudinal axis of said shaft.

51. The device of claim 42 further comprising said actuation wire extending through the elongate shaft, configured to translate, along a longitudinal axis of the elongate shaft, actuation of said effector.

52. The device of claim 42, wherein at least a portion of said clevis is characterized by a concaved-parabolic nozzle-like shape.

53. The device of claim 42, wherein at least a portion of said guide is characterized by a concaved-parabolic nozzle-like shape.

54. The device of claim 42 wherein at least one distal portion of said shaft comprises a coiled rotation-enabling shaft.

55. The device of claim 54, wherein a said flexible coiled rotation-enabling shaft is accommodated within said main flexible shaft.

56. The device of claim 54, wherein a distal part of said flexible coiled rotation-enabling shaft is accommodated within the clevis.

57. The device of claim 54, wherein said flexible coiled rotation-enabling shaft is connected with said link.

58. The device of claim 50, wherein a said actuator wire is accommodated within said coil system.

59. The device of claim 42, wherein is configured with an n-bar linkage, n is integer equal or greater than 3.

60. The device of claim 42, wherein parts of said device are flexible, rigid and any combination thereof.

61. The device of claim 42, wherein said device comprises a mechanism adapted to control at least three degrees of freedom, said degrees of freedom selected from a group consisting of activation of said element, articulation of distal end of said element; rotation of the distal end of said element, and any combination thereof.
